(19) United States
(12) Patent Application Publication
(10) Pub. No.: US 2013/0114785 A1
(43) Pub. Date: May 9, 2013

(54) METHOD FOR THE MEDICAL IMAGING OF A BODY PART, IN PARTICULAR THE HAND

(71) Applicants: Gerhard ALZEN, Heuchelheim (DE); Gabriel HARAS, Mucke (DE); Grzegorz SOZA, Heroldsberg (DE); Andreas WIMMER, Forchheim (DE)

(72) Inventors: Gerhard ALZEN, Heuchelheim (DE); Gabriel HARAS, Mucke (DE); Grzegorz SOZA, Heroldsberg (DE); Andreas WIMMER, Forchheim (DE)

(21) Appl. No.: 13/670,090

(22) Filed: Nov. 6, 2012

(30) Foreign Application Priority Data
Nov. 7, 2011 (DE) 102011085860.1

(51) Int. Cl. A61B 6/03

(52) U.S. Cl. 378/4

(57) ABSTRACT

A method is disclosed for the medical imaging of a body part, in particular the hand, wherein, in a tomographic image data record of the body part, points are marked which fix a face of interest running through the body part and which is singly or multiply curved. This curved face, which runs through all marked points, is then determined and the image content of the curved face is determined from the voxels of the image data record. This image content is mapped onto an observation plane which is finally displayed with the mapped image content on a screen. The method enables by way of example image representation of the structures of the left hand relevant to a skeleton examination by way of tomographic imaging.

### PRIORITY STATEMENT

The present application hereby claims priority under 35 U.S.C. §119 to German patent application number DE 10 2011 085 860.1 filed Nov. 7, 2011, the entire contents of which are hereby incorporated herein by reference.

### FIELD

At least one embodiment of the present invention generally relates to a method for the medical imaging of a body part, in particular the hand of a person.

### BACKGROUND

Imaging of the hand can be used by way of example for exact determination of the bone age or skeletal maturity. With the aid of an X-ray examination of the left hand, it is possible to determine the biological, physical maturity and to approximate the final body size of children or young people.

An X-ray examination of the left hand has previously been used as the standard method for determining skeletal maturity. Imaging takes place in a dorsoventral beam path using a film focus spacing of 1 m. The hand is positioned flat on the film cassette and with slightly spread-apart fingers and between thumb and index finger forms an angle of 30°. The presence of certain ossification centers and a maturity-related change in shape, the size of the bones and the course of the epiphyseal closure can be determined using this kind of X-ray.

Tomographic imaging techniques such as computerized tomography or magnetic resonance tomography can also be used for imaging the hand. However, these imaging methods have not previously been suitable for the above application since no single MPR representation (MPR: Multiplanar Reconstruction) is obtained from the sectional images on which all fundamental structures of the hand can be seen. Even with a huge expenditure of time a minimum of four to six images (depending on the positioning of the patient) have to be generated using the available MPR tools in order to display all relevant structures.

### SUMMARY

A method for medical imaging is disclosed with which a single image representation of the hand is made possible on which all structures required for determining the skeletal maturity can be seen.

Advantageous embodiments of the method are the subject matter of the dependent claims or may be inferred from the description and example embodiment below.

With an embodiment of the proposed method a tomographic image data record, hereinafter also called a 3D image data record, of the body part is produced using an imaging modality. In a suitable depiction of this 3D image data record points are then marked in this image data record which span a face of interest running through the body part and which is singly or multiply curved. The individual points can be marked by way of example by a user who causes individual layers of the 3D image data record to be displayed on the screen. The markings can be made using a suitable graphic input device, such as a mouse. The points in the tomographic image data record are in this connection taken to mean the individual image points of this 3D image data record or their spatial coordinates in the image data record.

Marking can also occur in automated fashion, as will be described in more detail below. A computer determines a curved face from the marked points of the 3D image data record, which face runs through all marked points and therefore, as a rule, is curved in all three dimensions. Depending on the position of the points this may be an open face or also an inherently closed face which then completely surrounds a certain volume. The image content is then determined or calculated for this curved face from the image points or voxels of the image data record, and this content corresponds to this curved face in the image data record. Grey-scale values are therefore allocated to the individual points of the curved surface which result from the grey-scale values of the image points of the 3D image data record which match or are directly adjacent to these points in terms of position.

An interpolation preferably occurs in this connection since the curved face often does not run exactly through the image points of the 3D image data record that are in a fixed grid. Once the image content of the curved face has been determined an observation plane is fixed and the image content of the curved face is mapped onto this observation plane. The observation plane is a plane face which can be oriented as desired relative to the image data record. This observation plane is then displayed on a screen with the mapped image content.

Any desired structures of the mapped body part not located in a plane may be visualized in a single image representation by way of the possibility provided by the present method of producing an MPR representation which is curved in all directions of the space and of displaying it on a screen mapped onto an observation plane. It is therefore possible, even in the case of tomographic images of the hand to display an overview of the individual bones in the hand from different levels of the image data record on a single 2D image.

To carry out at least one embodiment of the proposed method, a data processing device is preferably provided which processes the image acquisition data for the image representation obtained from the imaging modality according to the proposed method and displays the images required for marking the points and also the observation plane with the mapped image content on a screen. The data processing device is equipped with a suitable program for this purpose which executes the method steps following acquisition of the image data record and also enables interaction with the user.

### BRIEF DESCRIPTION OF THE DRAWINGS

The proposed method will be illustrated briefly again below with the aid of an example embodiment in conjunction with the drawings, in which:

FIGS. 1 and 2 show an example of the curved face, fixed in a 3D image data record of the hand, in two sections which are perpendicular to each other,

FIG. 3 shows the curved face fixed in FIG. 1 in a perspective view and

FIG. 4 shows a schematic view of an exemplary procedure of the proposed method.

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

With an embodiment of the proposed method a tomographic image data record, hereinafter also called a 3D image data record, of the body part is produced using an imaging modality. In a suitable depiction of this 3D image data record points are then marked in this image data record which span a face of interest running through the body part and which is singly or multiply curved. The individual points can be marked by way of example by a user who causes individual layers of the 3D image data record to be displayed on the screen. The markings can be made using a suitable graphic input device, such as a mouse. The points in the tomographic image data record are in this connection taken to mean the individual image points of this 3D image data record or their spatial coordinates in the image data record.

Marking can also occur in automated fashion, as will be described in more detail below. A computer determines a curved face from the marked points of the 3D image data record, which face runs through all marked points and therefore, as a rule, is curved in all three dimensions. Depending on the position of the points this may be an open face or also an inherently closed face which then completely surrounds a certain volume. The image content is then determined or calculated for this curved face from the image points or voxels of the image data record, and this content corresponds to this curved face in the image data record. Grey-scale values are therefore allocated to the individual points of the curved surface which result from the grey-scale values of the image points of the 3D image data record which match or are directly adjacent to these points in terms of position.

An interpolation preferably occurs in this connection since the curved face often does not run exactly through the image points of the 3D image data record that are in a fixed grid. Once the image content of the curved face has been determined an observation plane is fixed and the image content of the curved face is mapped onto this observation plane. The observation plane is a plane face which can be oriented as desired relative to the image data record. This observation plane is then displayed on a screen with the mapped image content.

Any desired structures of the mapped body part not located in a plane may be visualized in a single image representation by way of the possibility provided by the present method of producing an MPR representation which is curved in all directions of the space and of displaying it on a screen mapped onto an observation plane. It is therefore possible, even in the case of tomographic images of the hand to display an overview of the individual bones in the hand from different levels of the image data record on a single 2D image.

The proposed method of at least one embodiment thereby enables inter alia bone age determination to be carried out by way of tomographic imaging technology or using cross-sectional imaging techniques. The presence of a 3D image data record means regions located outside of the fixed curved face, for which additional imaging was necessary with the technology previously used, may also be visualized if required. Thus, in the case of the hand, by way of example, the sesamoid bone may also be depicted. The proposed method also enables a direct comparison of the sides of symmetrical objects, as is the case in particular with the skeletal system. Subsequent measurement of individual structures of the body part is also enabled or may be carried out more quickly with the proposed method owing to the three-dimensionally available data.

In a particularly advantageous embodiment, the tomographic image data record is created by means of magnetic resonance tomography. This has the particular advantage that the patient is not exposed to any damaging radiation due to X-rays.

In a development of the proposed method, marking of the points to fix the curved face can also occur by way of the computer itself or an algorithm which runs on it. For this purpose the corresponding body part is automatically segmented by way of example and by way of comparison with corresponding specified models the marking positions which are also indicated there are placed in the image data record. Automated techniques for segmenting body parts are known to the person skilled in the art.

In an advantageous embodiment of the proposed method a corresponding image representation according to the proposed method is shown to the user as early as after the marking of at least four points. In this image representation he can discern whether he can already see all structures of interest, or whether additional points must be placed in order to change the curved face. He may also be allowed to move individual points again, it being possible for him to interactively discern in each case the result of the action by way of the image representation changed thereby.

To carry out at least one embodiment of the proposed method, a data processing device is preferably provided which processes the image acquisition data for the image representation obtained from the imaging modality according to the proposed method and displays the images required for marking the points and also the observation plane with the mapped image content on a screen. The data processing device is equipped with a suitable program for this purpose which executes the method steps following acquisition of the image data record and also enables interaction with the user.

The proposed method will be illustrated again below with the aid of an example embodiment in which imaging of the left hand is carried out using magnetic resonance tomography (MRT).

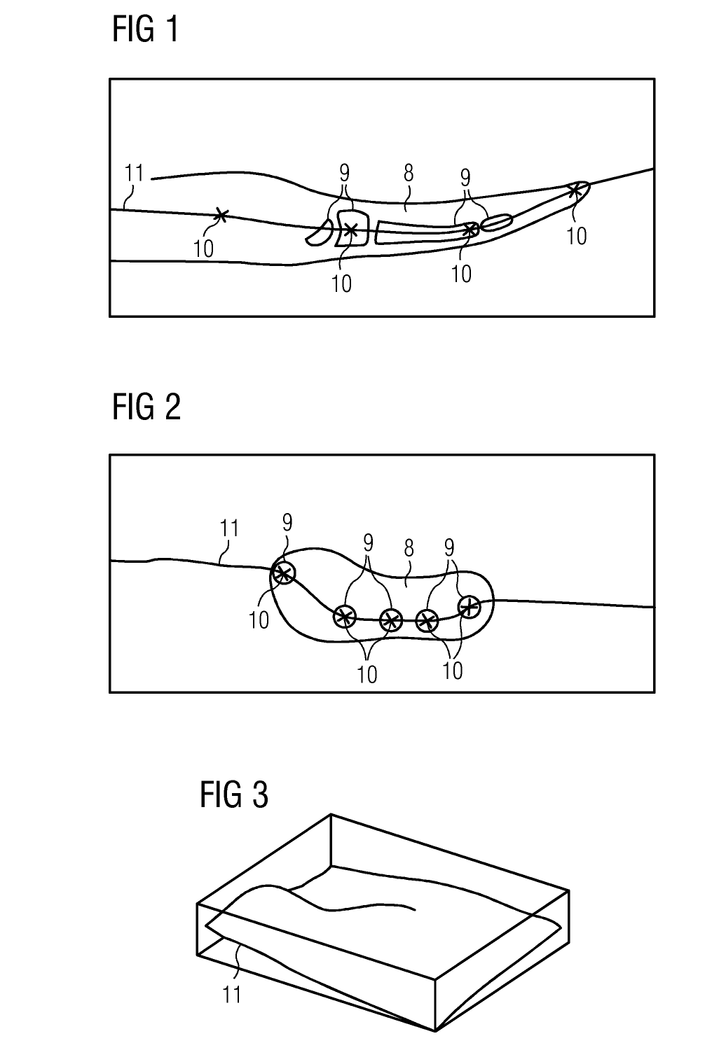

After the image has been acquired (step 1), in the 3D image data record obtained thereby, when displaying individual sectional images (step 2), markers are placed at the locations which will be of interest to the respective user during subsequent observation of the image (step 3). The markers can be placed in the image data record by user interaction, for example by clicking with a mouse. In the present example the user selects a plurality of sectional images of the hand in which he marks corresponding points. A curved face is then laid through the marker positions (step 4). This face or surface can be curved in all spatial directions. It is therefore a face which is singly or multiply curved. FIGS. 1 and 2 show by way of example in this regard two sectional images in mutually perpendicular planes through the hand 8 in which the bones of the hand 9, the marked points 10 and the curved face 11 calculated therefrom are shown. A perspective view of the curved face 11 can be seen in FIG. 3.

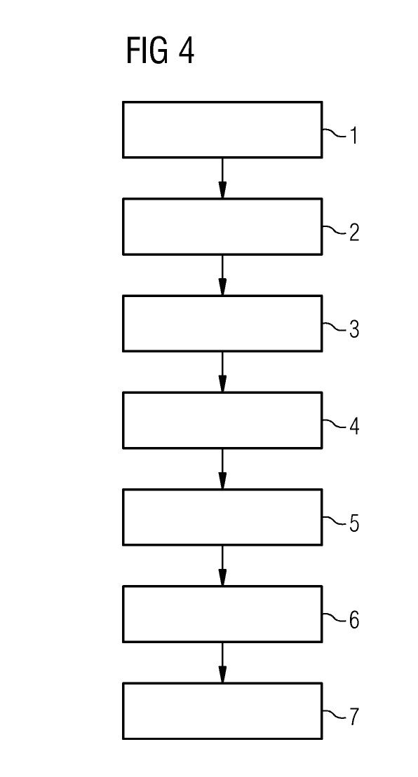

To create the image content, voxel values are interpolated (step 5) along the surface or curved face 11 and are finally projected (step 6) onto a 2D face for viewing or for further processing. The corresponding 2D face is then displayed on a screen with the projected image content (step 7). The view calculated using the method presented here is called a 3D curved MPR in accordance with the conventional term multi-planar reconstruction (MPR) for artificially calculated views in 3D image data records. The described example method steps 1-7 are schematized in FIG. 4.

The steps for determining the curved face and projection onto the 2D face will be illustrated in more detail below with the aid of examples again.

A 3D position (x, y, z) within the volume or image data record is allocated to each marker in accordance with the acquisition geometry associated with an MRT (or CT) volume data record. In step 4 an interpolating surface S is placed by way of these marker positions. If the markers describe a closed surface, then for example the method described in Carr, J. C. et al.: “Reconstruction and representation of 3d objects with radial basis functions, in SIGGRAPH '01: Proceedings of the 28th annual conference on Computer graphics and interactive techniques” (2001), pages 67-76, the entire contents of which are hereby incorporated herein by reference, for determining S can be used. For this it is necessary that normals to the surface S are also defined in addition to the markers.

In the application at hand here S is instead a height field h over the recording plane P which is assumed without limiting generality as being identical to the x/y plane. Depending on the application P can by way of example also be a standardized view (axial, sagittal or coronal) or else be oriented freely in the space as desired.

The height field h(x,y) and therewith S={(x, y, z)|(x, y)εP [symbol] z=h(x, y)} is given as a Thin-Plate Spline (TPS) surface by way of the markers and is calculated according to

$$h ( x ) = {\sum }_{i = 1}^{N} {λ}_{i} φ ( \vert x - {m}_{i} \vert ) ,$$

where x=(x,y), mi=(xi,yi) is the projection of the ith marker onto the plane P, N indicates the number of markers and |.| denotes the Euclidean distance. Details on the Thin-Plate Spline (TPS) can be found in Duchon, J.: “Splines minimizing rotation-invariant seminorms in Sobolev spaces”, in: W. Schempp and K. Zeller, editors, Constructive Theory of Functions of Several Variables, number 571 in Lecture Notes in Mathematics, Springer, (1977) pages 85-100, the entire contents of which are hereby incorporated herein by reference.

Used as the core φ(r) is φ(r)=r² log r, where log r provides the logarithm of the argument. Reference is made to the above publication by Carr, J. C. et al for tips on calculating the interpolation coefficient λᵢ.

After calculation of surface S, voxel values of the volume data record are determined in step 5 for positions (x,y,z)εS by interpolation. For display and further processing as a 2D view the interpolated voxel values of S are finally projected in step 6 onto an observation plane Q(i,j). Distortions inevitably occur in this connection. If these distortions do not matter, an orthographic projection can be performed in the above-described case of the height field, in which i=x and j=y and all z values are discarded. If S is a closed surface or if certain features, such as angles of the projection, are unaffected, more complex methods can be used for mapping onto the observation plane. The method described in Haker, S. et al: “Conformal Surface Parameterization for Texture Mapping”, in: IEEE Transactions on Visualization and Computer Graphics. vol. 6, No. 2 (2000), pages 181-189, the entire contents of which are hereby incorporated herein by reference, is suitable for this purpose by way of example for obtaining angles in the projection of S after Q.

In addition to the 3D curved MPR view already described it is also possible to calculate an entire 3D curved MPR volume. For an open surface S (height field), S is shifted several times in the positive and negative directions of the normals of P and a 3D curved MPR view is produced in each case. In the case of a closed surface 3D curved MPR views are calculated for surfaces S, which result due to inflation/deflation of S. The 3D curved MPR volume results in both cases as a totality of the individual 3D curved MPR views.

Although the invention has been illustrated and described in detail by the preferred example embodiment it is not limited by the disclosed examples. The person skilled in the art may derive other variations herefrom without departing from the scope of the invention.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims.

Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for the medical imaging of a body part, the method comprising:
acquiring a tomographic image data record of the body part;
marking points in the tomographic image data record which span a face of interest running through the body part and which is singly or multiply curved;
determining the curved face which runs through all marked points;
determining an image content of the curved face including image data from voxels of the image data record, through which the curved face runs;
fixing an observation plane and mapping the image content of the curved face onto the observation plane; and
displaying the observation plane with the mapped image content on a screen.

2. The method of claim 1, wherein the curved face is approximated using one or more functions.

3. The method of claim 1, wherein the curved face is approximated using a height function.

4. The method of claim 1, wherein the determining of the image content of the curved face occurs by interpolation from the image data of the image data record.

5. The method of claim 1, further comprising:
mapping of the image content of the curved face onto the observation plane occurs by way of parallel projection.

6. The method of claim 5, wherein angle-obtaining mapping is used for mapping the image content of the curved face onto the observation plane.

7. The method of claim 1, wherein the body part is a hand and wherein, for medical imaging of the hand, the marked points are selected in such a way that the curved face runs through the individual bones of the hand.

8. The method of claim 1, wherein the tomographic image data record is produced by way of MRT.

9. The method of claim 6, wherein a first depiction of the observation plane with the mapped image content is made on the screen as early as after marking at least four points, the first depiction, with subsequent marking of further points or interactive displacement of points already marked, being constantly adjusted by a user on the basis of a curved face changed thereby.

10. The method of claim 1, wherein the method is for the medical imaging of a hand.

11. The method of claim 2, wherein the curved face is approximated using one or more radial basis functions.

12. The method of claim 3, wherein the curved face is approximated using a Thin-Plate Spline (TPS) surface.

13. The method of claim 1, wherein angle-obtaining mapping is used for mapping the image content of the curved face onto the observation plane.

14. The method of claim 6, wherein the body part is a hand and wherein, for medical imaging of the hand, the marked points are selected in such a way that the curved face runs through the individual bones of the hand.

15. The method of claim 2, wherein the body part is a hand and wherein, for medical imaging of the hand, the marked points are selected in such a way that the curved face runs through the individual bones of the hand.
